(19) United States
(12) Patent Application Publication
(10) Pub. No.: US 2024/0218032 A1
(43) Pub. Date: Jul. 4, 2024

(54) NOVEL HUMAN PROGRAMMED DEATH LIGAND 1 (PD-L1) SPECIFIC BINDING MOLECULES

(71) Applicant: Navigo Proteins GmbH, Halle/Saale (DE)

(72) Inventors: Eva Bosse-Doenecke, Halle/Saale (DE); Manja Gloser-Braeunig, Halle/Saale (DE); Mathias Kahl, Halle/Saale (DE); Hanna Bobolowski, Halle/Saale (DE); Jonathan Lotze, Halle/Saale (DE)

(73) Assignee: Navigo Proteins GmbH, Halle/Saale (DE)

(21) Appl. No.: 18/556,860

(22) PCT Filed: Apr. 22, 2022
(86) PCT No.: PCT/EP2022/060712

§ 371 Date: Oct. 23, 2023

(30) Foreign Application Priority Data
Apr. 23, 2021 (EP) 21170116.4

(51) Int. Cl. C07K 14/47; A61K 47/64; A61K 49/00; A61K 51/08; G01N 33/569

(52) U.S. Cl. CPC C07K 14/4703; A61K 47/64; A61K 49/0056; A61K 49/006; A61K 51/08; G01N 33/56966

(57) ABSTRACT

The present invention relates to new ubiquitin derived molecules that are specific for Programmed Death Ligand 1 (PD-L1). The PD-L1 specific molecules of the invention inhibit the interaction between PD1 and PD-L1. The invention further refers to PD-L1 specific Affilin® proteins that further comprise a diagnostically or therapeutically active component. Further aspects of the invention cover the use of these PD-L1 binding proteins in medicine, for example, in diagnosis and therapy of PD-L1 related cancer.

### FIELD OF THE INVENTION

The present invention relates to new ubiquitin derived molecules that are specific for Programmed Death Ligand 1 (PD-L1). The PD-L1 specific molecules of the invention inhibit the interaction between PD-1 and PD-L1. The invention further refers to PD-L1 specific Affilin® proteins that further comprise a diagnostically or therapeutically active component. Further aspects of the invention cover the use of these PD-L1 binding proteins in medicine, for example, in diagnosis and therapy of PD-L1 related cancer.

### BACKGROUND OF THE INVENTION

The transmembrane protein Programmed Death Ligand 1 (PD-L1; also known as B7-H, B7H1, CD274) belongs to B7 family of immune regulatory molecules and is involved in the regulation of cellular and humoral immune responses. The PD-L1 ligand interacts with PD-1 (programmed cell death 1). PD-L1 is expressed on macrophages, T cells, and B cells, keratinocytes, enothelial and intestinal epithelial cells, as well as a variety of carcinomas and melanoma.

Both PD-1 and PD-L1 belong to the family of immune checkpoint proteins that act as co-inhibitory factors. They can halt or limit the development of the T cell response, for example suppress T cell activation and proliferation and induce the apoptosis of activated T cells. The PD-1/PD-L1 interaction ensures that the immune system is activated only at the appropriate time in order to minimize the possibility of chronic autoimmune inflammation.

PD-L1 is overexpressed on tumor cells; PD-L1 occurs in dimerized form. PD-L1 binds to PD-1 receptors on the activated T cells, which leads to the inhibition of the cytotoxic T cells and thereby to reduced cytokine production and proliferation of T cells. Through up-regulation of PD-L1 expression, tumor cells escape detection and destruction by the immune system. In cancer, PD-L1 provides resistance to T cell mediated lysis. By blocking checkpoint inhibitors including PD-1 and PD-L1, the immune system can overcome the cancer's ability to resist the immune responses and stimulate the body's own mechanisms to remain effective in its defenses against cancer. The inhibition of dimerization of PD-L1 blocks the interaction between PD-L1 and PD-1.

Several immune check point humanized IgG, antibodies were developed as inhibitors for immunotherapy, for example, Atezolizumab, Durvalumab, Avelumab, BMS-936559, MEDI4736.

Diagnosis and treatment of PD-L1 related cancer is not adequately addressed by existing options, and as a consequence, many patients do not adequately benefit from current strategies.

Needless to say that there is a strong need for novel strategies for diagnosis and treatment of tumors with PD-L1 overexpression.

One objective of the present invention is the provision of molecules for specific targeting of PD-L1 for allowing targeted diagnostic and treatment options, including detection of PD-L1 positive tumors (e.g. by radio-diagnostic methods). Targeting this tumor-associated protein may offer benefit to patients with unmet need for novel diagnostic and therapeutic routes. Targeting PD-L1 suggests potentially non-toxic diagnostic and treatment approach, due to low and restricted distribution of PD-L1 in normal tissues. Thus, binding proteins with specificity for PD-L1 may enable effective medical options for cancer, and finally improve quality of life for patients.

The invention provides novel PD-L1 binding molecules for new and improved strategies in the diagnosis and treatment of PD-L1 related cancer. Further, the novel PD-L1 binding molecules of the invention provide improved strategies in the diagnosis and treatment of infectious diseases such as chronic viral infection.

The above-described objectives and advantages are achieved by the subject-matter of the appended claims. The present invention meets the needs presented above by providing examples for PD-L1 binding proteins. The above overview does not necessarily describe all problems solved by the present invention.

### SUMMARY OF THE INVENTION

The present disclosure provides the following items 1 to 15, without being specifically limited thereto:

1. A protein comprising an amino acid sequence of at least 90% identity to any one selected from the group of SEQ ID NOs: 1-20, wherein the protein has a specific binding affinity for human Programmed Death Ligand 1 (PD-L1). All proteins with binding affinity for PD-L1 are ubiquitin muteins with less than 91% identity to ubiquitin (SEQ ID NO: 21). Ubiquitin does not bind to PD-L1. All proteins bind to the same or overlapping epitope of PD-L1 and wherein the protein inhibits the interaction of PD-L1 with PD-1.

2. The protein according to item 1, wherein protein has a binding affinity for human PD-L1 of at least 100 nM.

3. The protein according to items 12 wherein the protein is stable in serum after 24 h incubation at 37° C.

4. The protein according to any one of items 1-3 wherein the protein is a multimer comprising of a plurality of the proteins according to any one of items 1-3, preferably a dimer, a trimer, or a tetramer of the protein according to any one of items 1-3.

4. The protein according to any one of items 1-3, further comprising one or more coupling sites for the coupling of chemical moieties, preferably wherein the chemical moieties are selected from any of chelators, drugs, toxins, dyes, and small molecules.

5. The protein according to any one of items 1-4, further comprising at least one diagnostically active moiety, optionally selected from a radionuclide, fluorescent protein, photosensitizer, dye, enzyme, magnetic beads, metallic beads, colloidal particles, electron-dense reagent, biotin, digoxigenin, hapten, CAR-T, or exosomes, or any combination thereof.

6. The protein according to any one of items 1-4, further comprising at least one therapeutically active moiety, optionally selected from a monoclonal antibody or a fragment thereof, a binding protein, a receptor or receptor domain, a receptor ligand, a radionuclide, a cytotoxic compound, a cytokine, a chemokine, an enzyme, CAR-T, or exosomes, or derivatives thereof, or any combination of the above.

7. The protein according to any one of items 1-6, further comprising at least one moiety modulating pharmacokinetics selected from optionally a serum albumin, an albumin-binding protein, an immunoglobulin binding protein, or an immunoglobulin or immunoglobulin fragment, a polysaccharide, an unstructured amino acid sequence comprising amino acids alanine, glycine, serine, proline, a polyethylene glycol, a sialic acid, or a transferrin.

8. The protein according to any one of items 1-7, for use in diagnosis or treatment of PD-L1 related tumors, or infectious diseases such as chronic viral infection (HIV, HBV, HCV).

9. A method of diagnosis or treatment of PD-L1 related tumors, or infectious diseases such as chronic viral infection (HIV, HBV, HCV), comprising administering to a subject in need thereof the protein according to any one of items 1-7. In various embodiments, the method comprises the administration of a composition comprising the protein according to any one of items 1-7. The protein, or a composition comprising the protein, is administered to the subject in a diagnostically or therapeutically effective amount thereof. Preferably, the subject is a mammalian subject. More preferably, the mammalian subject is a human subject. In various embodiments, the mammalian subject is an animal, including but not limited to cows, sheep, rabbit, pig, mouse, rat, goat, cat, dog, and the like. Preferably, the animal is a pet, including but not limited to a cat, dog, mouse, and the like.

10. A composition comprising the protein according to any one of items 1-7 for use in medicine, preferably for use in the diagnosis or treatment of PD-L1 related tumors or infectious diseases such as chronic viral infection.

11. A method of producing the protein according to any one of items 1-7, comprising the steps of a) culturing a host cell under conditions suitable to obtain said protein and b) isolating said protein produced.

12. A method of detecting PD-L1 comprising a sample with a protein according to any of items 1-7, and detecting binding of PD-L1 with a protein according to any of items 1-7 by contacting the sample with proteins according to any of items 1-7.

This summary does not necessarily describe all features of the present invention. Other embodiments come apparent from a review of the ensuing detailed description.

### BRIEF DESCRIPTION OF THE FIGURES

The Figures show:

FIG. 1: shows selected amino acid sequences of PD-L1 binding proteins compared to wildtype ubiquitin (SEQ ID NO: 21). The PDL-1 specific Affilin® proteins have identities lower than 91% to ubiquitin. The numbers in the top row refer to the corresponding amino acid position. Positions that are identical to ubiquitin are highlighted in grey; differences are shown in white. Not shown in this table is the additional insertion of 4 or 6 amino acids between position 9 and 10 in SEQ ID NO: 1 (215306); SEQ ID NO: 2 (215220), SEQ ID NO: 3 (211904), and SEQ ID NO: 4 (215208).

FIG. 2: shows the stability of PD-L1 binding proteins in human serum and mouse serum. KD values vs PD-L1 on overexpressing cells were determined in PBS (circles) and after 24 h incubation at 37° C. in mouse serum (triangle). After 24 h incubation in serum, the KD value showed only minor variation compared to the value before incubation in serum, assuming that the PDL-1 specific Affilin is stable under these conditions. The stability of Affilin®215208 in mouse serum is shown in FIG. 2A and in human serum in FIG. 2B. The stability of Affilin®215220 in mouse serum is shown in FIG. 2C and in human serum in FIG. 2D.

FIG. 3: shows an analysis of binding of Affilin®211904 with high affinity to PD-L1-Fc (label-free interaction assays using SPR (Biacore)). PD-L1 was immobilized on a CM-5 chip. After fitting the data with a 1:1 langmuir model a K_{D} value of 3.6 nM was calculated.

FIG. 4: shows an analysis of competitive binding of Affilin® proteins to hPD-1/hPD-L1-Fc (analysis via label-free interaction assays using SPR (Biacore)). FIG. 4A. Analysis of competitive binding of Affilin®211828 and Affilin®211883 to hPD-1/hPD-L1-Fc shows that the addition of Affilin® leads to significant reduced binding of hPD-L1-Fc to hPD-1. Similar results were obtained for Affilin®211904. FIG. 4B. Analysis of competitive binding of Affilin®211828 and Affilin®211883 to mPD-1/mPD-L1-Fc shows that the addition of Affilin leads to significant reduced binding of mPD-L1-Fc to mPD-1.

FIG. 5: shows an label-free interaction assay (SPR) of competitive binding of Affilin® 211904 to hPD-L1-Fc1. hPD-L1-Fc1 was immobilized. The dotted line shows 211904+hPD-1, the black line shows PBST+hPD-1. The figure shows that the addition of Affilin®211904 leads to significant reduced binding of hPD-L1-Fc to hPD-1.

FIG. 6: shows an label-free interaction assays (SPR) of competitive binding of Affilin®211904 to hPD-L1-Fc and shows that the addition of Affilin®211904 leads to significant reduced binding of hPD-L1-Fc to Affilin®211620. The dotted line shows 211904+211620, the black line shows PBST+211620. The figure shows that the addition of Affilin®211904 leads to significant reduced binding of Affilin®211620 to hPD-L1-Fc; thus Affilin®211904 and Affilin®211620 compete for the same or an overlapping epitope on PD-L1. Further results with other PD-1-binding Affilin® proteins are shown in Table 4 (see Example 7).

### DETAILED DESCRIPTION OF THE INVENTION

The present inventors have developed a solution to meet the strong ongoing need in the art for expanding medical options for the diagnosis and treatment of cancer and infectious diseases by providing novel PD-L1 binding proteins. The PD-L1 specific proteins as defined herein are functionally characterized by high specific affinity for PD-L1; all PD-L1 specific proteins of the invention bind to the same or overlapping epitope of PD-L1. Further, they show high levels of stability, both in serum and at high temperatures. The PD-L1 binding proteins of the invention inhibit the interaction of PD-L1 with PD-1. The invention provides PD-L1 binding proteins based on ubiquitin muteins (also known as Affilin® molecules). The PD-L1 binding proteins as described herein thereby provide molecular formats with favorable physicochemical properties, high-level expression in bacteria, and allow easy production methods. The novel PD-L1 binding proteins may broaden so far unmet medical strategies for the diagnosis and therapy of PD-L1 related cancer and infectious diseases. In particular, the PD-L1 binding proteins may be used for imaging purposes, for example, for the presence of tumor cells expressing PD-L1, and for radiotherapy treatment of tumors expressing PD-L1, or for immunooncological treatment options.

Before the present invention is described in more detail below, it is to be understood that this invention is not limited to the particular methodology, protocols and reagents described herein as these may vary. It is also to be understood that the terminology used herein is for the purpose of describing particular aspects and embodiments only and is not intended to limit the scope of the present invention which is reflected by the appended claims. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new target-specific binding molecules for use in technical applications and in therapy and diagnostics.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims, which follow, unless the context requires otherwise, the word “comprise”, and variants such as “comprises” and “comprising”, was understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, GenBank Accession Number sequence submissions etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing that, with its whole content and disclosure, forms part of the disclosure content of the present specification.

### General Definitions of Important Terms Used in the Application

The term “PD-L1” as used herein refers to Uniprot accession number Q9NZQ7 (programmed cell death 1 ligand 1). The term “PD-L1” comprises all polypeptides which show a sequence identity of at least 70%, 75%, 80%, 85%, 90%, 95%, 96% or 97% or more, or 100% to the PD-L1 of Uniprot accession number Q9NZQ7 (human). The human PD-L1 is 73% identical zu mouse PD-L1 (accession number Q9EP73) and 74% identical to rat PD-L1. The term “PD-L1” includes the extracellular domain (residues 19-238) of PD-L1. Human PD-L1 possesses three domains: an extracellular domain (aa 19-238), a transmembrane domain (aa 239-259), and a cytoplasmic domain (aa 260-290). The extracellular domain of human PD-L1 is shown in SEQ ID NO: 26.

The terms “PD-L1 binding protein” or “PD-L1 specific Affilin® protein” refer to a protein with high affinity binding to PD-L1. Accordingly, the proteins of the present disclosure are PD-L1 binding proteins. As described elsewhere herein, the proteins of the present disclosure exhibit a specific binding affinity for human PD-L1, which is a binding affinity in the nanomolar range. In various embodiments, a PD-L1 binding protein of the present invention is a protein comprising an amino acid sequence of at least 90% identity to any one selected from the group of SEQ ID NOs: 1 to 20, wherein the protein has a binding affinity for human PD-L1 of at least (or less than) 250 nM, and wherein the protein inhibits the interaction of PD-L1 with PD-1. In accordance with Table 3, a PD-L1 binding protein of the present invention may have a binding affinity for human PD-L1 of at least (or less than) 220 nM.

The term “Affilin®” is a registered trademark of Navigo Proteins® GmbH and refers to non-immunoglobulin derived binding proteins.

The terms “protein” and “polypeptide” refer to any chain of two or more amino acids linked by peptide bonds, and does not refer to a specific length of the product. Thus, “peptides”, “protein”, “amino acid chain”, or any other term used to refer to a chain of two or more amino acids, are included within the definition of “polypeptide”, and the term “polypeptide” may be used instead of, or interchangeably with, any of these terms. The term “polypeptide” is also intended to refer to the products of post-translational modifications of the polypeptide, which are well known in the art.

The term “modification” or “amino acid modification” refers to a substitution, a deletion, or an insertion of a reference amino acid at a particular position in a parent polypeptide sequence by another amino acid. Given the known genetic code, and recombinant and synthetic DNA techniques, the skilled scientist can readily construct DNAs encoding the amino acid variants.

The term “ubiquitin” refers to the amino acid sequence given in SEQ ID NO: 21 and to proteins with at least 95% identity, such as for example with point mutations in positions 45, 75, 76 which do not influence binding to a target (e.g. PD-L1).

The term “mutein” as used herein refers to derivatives of, for example, ubiquitin as shown in SEQ ID NO: 21, which differ from said amino acid sequence by amino acid exchanges, insertions, deletions or any combination thereof, provided that the mutein has a specific binding affinity to PD-L1.

The term “substitution” is understood as exchange of an amino acid by another amino acid. The term “insertion” comprises the addition of amino acids to the original amino acid sequence. The terms “binding affinity” and “binding activity” may be used herein interchangeably, and they refer to the ability of a polypeptide to bind to another protein, peptide, or fragment or domain thereof. Binding affinity is typically measured and reported by the equilibrium dissociation constant (K_{D}), which is used to evaluate and rank order strengths of bimolecular interactions.

The term “fusion protein” relates to a protein comprising at least a first protein joined genetically to at least a second protein. A fusion protein is created through joining of two or more genes that originally coded for separate proteins. Fusion proteins may further comprise additional domains that are not involved in binding of the target, such as but not limited to, for example, multimerization moieties, polypeptide tags, polypeptide linkers or moieties binding to a target different from PD-1.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide. Methods for sequence alignment are well known in the art. For example, for determining the extent of an amino acid sequence identity of an arbitrary polypeptide relative to another amino acid sequence, the SIM Local similarity program as known in the art is preferably employed. For multiple alignment analysis, Clustal Omega is preferably used, as known to someone skilled in the art.

### DETAILED DESCRIPTION OF THE EMBODIMENTS OF THIS INVENTION

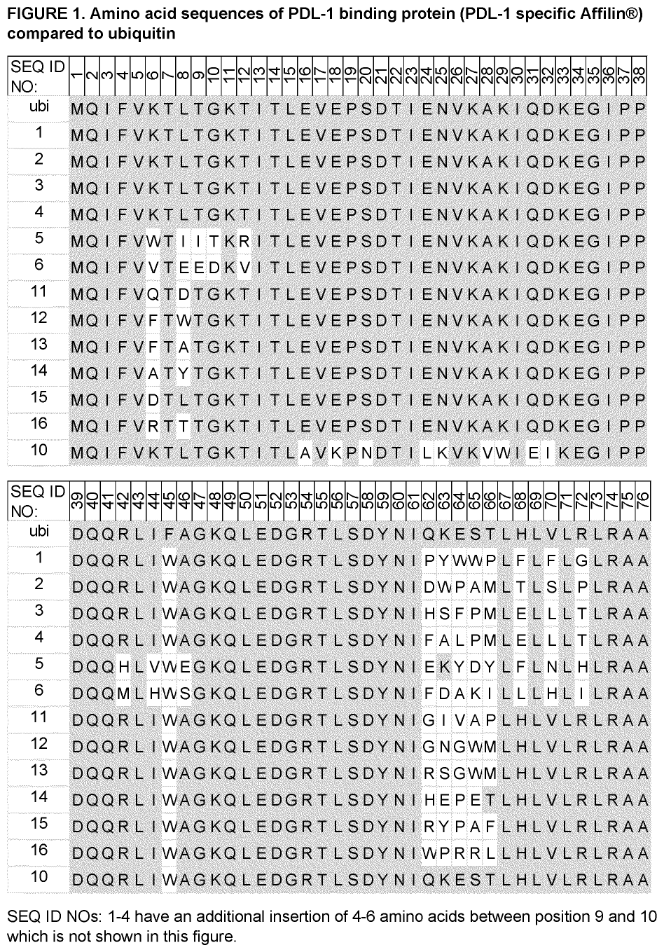

Structural characterization of PD-L1 binding proteins. The PD-L1 binding protein as described herein comprises, essentially consists, or consists of an amino acid sequence selected from any one of the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14, SEQ ID NO: 15, SEQ ID NO: 16, SEQ ID NO: 17, SEQ ID NO: 18, SEQ ID NO: 19, SEQ ID NO: 20, or the PD-L1 binding protein is selected from amino acid sequences with at least 90% identity thereto, respectively. In various embodiments, the PD-L1 binding protein is selected from amino acid sequences with at least 91%, 92%, 93%, 94%, or 95%, or even 96%, 97%, 98%, or 99% identity to the respective amino acid sequences of SEQ ID NOs: 1 to 20. Selected PD-L1 binding proteins are shown in FIG. 1. All PD-L1 binding proteins are based on ubiquitin and share the same basic scaffold (see FIG. 1) and are less than 91% identical to ubiquitin (SEQ ID NO: 21). All PD-L1 binding proteins described herein comprise at least one monomer of at least 76 amino acid having amino acids 1M, 2Q, 31, 4F, 5V, 7T, 11K, 131, 14T, 15L, 17V, 19P, 21D, 22T, 231, 26V, 27K, 301, 33K, 34E, 35G, 361, 37P, 38P, 39D, 40Q, 41Q, 43L, 47G, 48K, 49Q, 50L, 51E, 52D, 53G, 54R, 55T, 56L, 57S, 58D, 59Y, 60N, 611, 67L, 69L, 71L, 73L, 74R, 75A, 76A. In some embodiments, modifications in ubiquitin that result in binding to PD-L1 are located between amino acids in region 6-12, in region 42-46, and/or in region 62-72 of ubiquitin (SEQ ID NO: 21). In some embodiments, the PD-L1 specific ubiquitin muteins have additional insertions of 4-6 amino acids between position 9-10 (for example, SEQ ID NOs: 1-4). In one embodiment, modifications are the alpha-helical region of ubiquitin resulting in PD-L1 binding (for example, SEQ ID NO: 10).

In accordance with FIG. 1, in various embodiments of the present invention, the PD-L1 binding protein has at least 90% sequence identity to the amino acid sequence of any one of SEQ ID NOs: 1 to 4 and comprises, at positions corresponding to positions 45, 62-66, 68, 70, and 72 of the respective SEQ ID NO: 1, 2, 3, or 4, one or more of the amino acid residues at said positions of the respective SEQ ID NO: 1, 2, 3, or 4 as shown in FIG. 1. As described elsewhere herein, the PD-L1 binding protein has a binding affinity for human PD-L1 of at least 250 nM, and inhibits the interaction of PD-L1 with PD-1. In preferred embodiments, the binding affinity for PD-L1 corresponds to the respective binding affinity for SEQ ID NOs: 1 to 4 shown in Table 3.

In accordance with FIG. 1, in various embodiments of the present invention, the PD-L1 binding protein has at least 90% sequence identity to the amino acid sequence of SEQ ID NO: 5 and comprises, at positions corresponding to positions 6, 8-10, 12, 42, 44-46, 62, 64-66, 68, 70, and 72 of SEQ ID NO: 5, one or more of the amino acid residues at said positions of SEQ ID NO: 5 as shown in FIG. 1. As described elsewhere herein, the PD-L1 binding protein has a binding affinity for human PD-L1 of at least 250 nM, and inhibits the interaction of PD-L1 with PD-1. In preferred embodiments, the binding affinity for PD-L1 corresponds to the binding affinity for SEQ ID NO: 5 shown in Table 3.

In accordance with FIG. 1, in various embodiments of the present invention, the PD-L1 binding protein has at least 90% sequence identity to the amino acid sequence of SEQ ID NO: 6 and comprises, at positions corresponding to positions 6, 8-10, 12, 42, 44-46, 62-66, 68, 70, and 72 of SEQ ID NO: 6, one or more of the amino acid residues at said positions of SEQ ID NO: 6 as shown in FIG. 1. As described elsewhere herein, the PD-L1 binding protein has a binding affinity for human PD-L1 of at least 250 nM, and inhibits the interaction of PD-L1 with PD-1. In preferred embodiments, the binding affinity for PD-L1 corresponds to the binding affinity for SEQ ID NO: 6 shown in Table 3.

In accordance with FIG. 1, in various embodiments of the present invention, the PD-L1 binding protein has at least 90% sequence identity to the amino acid sequence of any one of SEQ ID NOs: 11 to 13 and 16, and comprises, at positions corresponding to positions 6, 8, 45, and 62-66 of the respective SEQ ID NO: 11, 12, 13 or 16, one or more of the amino acid residues at said positions of the respective SEQ ID NO: 11, 12, 13 or 16 as shown in FIG. 1. As described elsewhere herein, the PD-L1 binding protein has a binding affinity for human PD-L1 of at least 250 nM, and inhibits the interaction of PD-L1 with PD-1.

In accordance with FIG. 1, in various embodiments of the present invention, the PD-L1 binding protein has at least 90% sequence identity to the amino acid sequence of SEQ ID NO: 14 and comprises, at positions corresponding to positions 6, 8, 45, and 62-65 of SEQ ID NO: 14, one or more of the amino acid residues at said positions of SEQ ID NO: 14 as shown in FIG. 1. As described elsewhere herein, the PD-L1 binding protein has a binding affinity for human PD-L1 of at least 250 nM, and inhibits the interaction of PD-L1 with PD-1.

In accordance with FIG. 1, in various embodiments of the present invention, the PD-L1 binding protein has at least 90% sequence identity to the amino acid sequence of SEQ ID NO: 15 and comprises, at positions corresponding to positions 6, 45, and 62-66 of SEQ ID NO: 15, one or more of the amino acid residues at said positions of SEQ ID NO: 15 as shown in FIG. 1. As described elsewhere herein, the PD-L1 binding protein has a binding affinity for human PD-L1 of at least 250 nM, and inhibits the interaction of PD-L1 with PD-1.

In accordance with FIG. 1, in various embodiments of the present invention, the PD-L1 binding protein has at least 90% sequence identity to the amino acid sequence of SEQ ID NO: 10 and comprises, at positions corresponding to positions 16, 18, 20, 24, 25, 28, 29, 31, 32, and 45 of SEQ ID NO: 10, one or more of the amino acid residues at said positions of SEQ ID NO: 10 as shown in FIG. 1. As described elsewhere herein, the PD-L1 binding protein has a binding affinity for human PD-L1 of at least 250 nM, and inhibits the interaction of PD-L1 with PD-1. In preferred embodiments, the binding affinity for PD-L1 corresponds to the binding affinity for SEQ ID NO: 10 shown in Table 3.

In various preferred embodiments of the embodiments described above in relation to FIG. 1, the PD-L1 binding protein has at least 92%, 93%, 94%, or 95% sequence identity to the amino acid sequence of any one of SEQ ID NOs: 1 to 6 and 10 to 16, respectively. In various other preferred embodiments of the embodiments described above in relation to FIG. 1, the PD-L1 binding protein has at least 97% or 98% sequence identity to the amino acid sequence of any one of SEQ ID NOs: 1 to 6 and 10 to 16, respectively. Furthermore, in accordance with Table 3, in various preferred embodiments the PD-L1 binding protein may have a binding affinity for human PD-L1 of at least (or less than) 100 nM.

As described elsewhere herein, the PD-L1 binding proteins of the present invention bind to the same or overlapping epitope of PD-L1. In various embodiments, the PD-L1 binding protein of the present invention binds to the N-terminal region of the extracellular domain of PD-L1. In various preferred embodiments, the PD-L1 binding protein of the present invention binds to an N-terminal region of the extracellular domain of PD-L1 comprising amino acid residues 1 to about 50 of SEQ ID NO: 26 (corresponding to amino acid residues 19 to about 68 of the amino acid sequence of PD-L1). In various other preferred embodiments, the PD-L1 binding protein of the present invention binds to an N-terminal region of the extracellular domain of PD-L1 comprising amino acid residues 1 to about 40 of SEQ ID NO: 26 (corresponding to amino acid residues 19 to about 58 of the amino acid sequence of PD-L1). In still other preferred embodiments, the PD-L1 binding protein of the present invention binds to an N-terminal region of the extracellular domain of PD-L1 comprising amino acid residues 1 to about 30 of SEQ ID NO: 26 (corresponding to amino acid residues 19 to about 48 of the amino acid sequence of PD-L1).

In particularly preferred embodiments, the PD-L1 binding protein of the present invention binds to an N-terminal region of the extracellular domain of PD-L1 comprising amino acid residues 1 to 15 of SEQ ID NO: 26 (corresponding to amino acid residues 19 to 33 of the amino acid sequence of PD-L1). In even more preferred embodiments of the present invention, the PD-L1 binding protein binds to an epitope in the N-terminal region of the extracellular domain of PD-L1 as described above, wherein the epitope comprises, or consists of, amino acid residues 9 to 15 of SEQ ID NO: 26 (corresponding to amino acid residues 27 to 33 of the amino acid sequence of PD-L1). In various embodiments, a PD-L1 binding protein of the present invention having at least 90% sequence identity to the amino acid sequence of any one of SEQ ID NOs: 10, 15 and 16 as described elsewhere herein binds to said epitope comprising, or consisting of, amino acid residues 9 to 15 (“LYVVEYG”) of SEQ ID NO: 26.

In various other embodiments, a PD-L1 binding protein of the present invention having at least 90% sequence identity to the amino acid sequence of any one of SEQ ID NOs: 8, 9, and 17 to 20 described elsewhere herein binds to said epitope comprising amino acid residues 9 to 15 (“LYVVEYG”) of SEQ ID NO: 26.

In other preferred embodiments, the PD-L1 binding protein of the present invention binds to an N-terminal region of the extracellular domain of PD-L1 comprising amino acid residues 25 to 47 of SEQ ID NO: 26 (corresponding to amino acid residues 43 to 65 of the amino acid sequence of PD-L1). In various embodiments, a PD-L1 binding protein of the present invention having at least 90% sequence identity to the amino acid sequence of any one of SEQ ID NOs: 8, 9, 10, and 15 to 20 as described elsewhere herein binds to an epitope in the N-terminal region of the extracellular domain of PD-L1 as described above, wherein the epitope comprises, or consists of, amino acid residues 33 to 39 (“AALIVYW”) of SEQ ID NO: 26 (corresponding to amino acid residues 51 to 57 of the amino acid sequence of PD-L1).

As further described herein, a PD-L1 binding protein of the present invention including multimeric forms may bind to one or more epitopes in the N-terminal region of the extracellular domain of PD-L1. In various embodiments of the present invention, a multimeric form of a PD-L1 binding protein of the present invention, preferably a dimer, may bind to both of the above-identified epitopes comprising, or consisting of, the amino acid sequences “LYVVEYG” and “AALIVYW” in the N-terminal region of the extracellular domain of PD-1.

Functional characterization. In some embodiments, the PD-L1 binding protein as described herein has a binding affinity (K_{D}) of less than (or at least) 200 nM for PD-L1. The PD-L1 binding proteins bind PD-L1 with measurable binding affinity of less than 200 nM, less than 100 nM, less than 50 nM, less than 20 nM, and less than 10 nM. In accordance with Table 3, in various embodiments, the PD-L1 binding proteins bind PD-L1 with measurable binding affinity of at least (or less than) 220 nM. The appropriate methods are known to those skilled in the art or described in the literature. The methods for determining the binding affinities are known per se and can be selected for instance from the following methods known in the art: enzyme-linked immunosorbent assay (ELISA), surface plasmon resonance (SPR), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Some of the methods are described in the Examples below. Typically, the dissociation constant KD is determined at 20° C., 25° C., or 30° C. If not specifically indicated otherwise, the KD values recited herein are determined at 25° C. by SPR. The lower the K_{D} value, the greater the binding affinity of the biomolecule for its binding partner. The higher the K_{D} value, the more weakly the binding partners bind to each other (see Examples).

In some embodiments, the PD-L1 binding protein as described herein are particularly stable under different conditions.

In some embodiments, the PD-L1 binding proteins are stable in the presence of serum for at least 24 h at 37° C. In some embodiments, the PD-L1 binding proteins are stable in the presence of human serum for at least 24 h at 37° C., as described in Examples in more detail. In some embodiments, the PD-L1 binding proteins are stable in the presence of mouse serum for at least 24 h at 37° C. For example, the stability of a PD-L1 binding protein can be determined by measuring the binding affinity (K_{D}) after incubation in serum for a long period of time at temperatures as high as 37° C. using standard methods as described herein above and in the Examples.

In some embodiments, the PD-L1 binding protein as described herein is stable at high temperatures, preferably of at least 60° C., more preferably of at least 62° C. For stability analysis, for example spectroscopic or fluorescence-based methods in connection with chemical or physical unfolding are known to those skilled in the art. For example, the stability of a molecule can be determined by measuring the thermal melting (Tₘ) temperature, the temperature in ° Celsius (° C.) at which half of the molecules become unfolded, using standard methods. Typically, the higher the Tₘ, the more stable the molecule. Temperature stability was determined by differential scanning fluorimetry (DSF), as described in further detail in Example 4 and in Table 3.

In some embodiments, comparing PD-L1 binding proteins show that PD-L1 binding proteins may bind non identical or non-overlapping epitopes (see Example 7). All PD-L1 binding proteins of the invention bind to the same or overlapping epitope. In addition, the competition of the PD-L1 binding proteins for the PD-L1/PD-1 interface was tested. All PD-L1 binding proteins inhibited the interaction of PD-1 binding to PD-L1 (e.g. Example 6). PD-L1 binding proteins of the invention bind specifically to PD-L1 and inhibit thereby the interaction of PD-1 with PD-L1.

In some embodiments, the specific binding of PD-L1 specific Affilin® proteins confirmed by cellular PD-L1 binding analysis with overexpressing cells. Cellular PD-L1 binding of the PD-L1 specific Affilin® proteins can be determined by standard methods, including Immunofluorescence microscopy and flow cytometric analysis. The Examples confirm the specific binding of PD-L1 binding proteins as described herein on PD-L1 expressing cells (see Examples).

Multimers. In some embodiments, the PD-L1 binding protein is a multimer comprising of a plurality of the PD-L1 binding protein as defined herein. A multimer may comprise two, three, four, or more PD-L1 binding proteins. In one embodiment, the PD-L1 binding protein comprises 2, 3, 4, or more PD-L1 binding proteins linked to each other, i.e. the PD-L1-binding protein can be a dimer, trimer, or tetramer, etc.

For example, the PD-L1 binding protein of SEQ ID NO: 7 is a dimer (Affilin®211960) having SEQ ID NO: 11 as N-terminal monomer and SEQ ID NO: 12 as C-terminal monomer. The PD-L1 binding protein of SEQ ID NO: 8 is a dimer (Affilin®211944) having SEQ ID NO: 13 as N-terminal monomer and SEQ ID NO: 14 as C-terminal monomer. The PD-L1 binding protein of SEQ ID NO: 9 is a dimer (Affilin®211828) having SEQ ID NO: 15 as first (N-terminal) monomer and SEQ ID NO: 16 as second (C-terminal) monomer.

In some embodiments, two or more PD-L1 binding proteins are directly linked. In some embodiments, two or more PD-L1 binding proteins are linked by a peptide linker. In various embodiments, two or more PD-L1 binding proteins are linked via a peptide linker of up to 30 amino acids. In other embodiments, two or more PD-L1 binding proteins are linked via a peptide linker of 10, 11, 12, 13, 14, 15, 16, 17, 18, 19, 20 amino acids. Selected examples for multimers are shown in in Table 1.

TABLE 1
Multimeric PD-L1 binding proteins

| SEQ ID | | | |
|---|---|---|---|
| NO: | Affilin ® | Multimer of | Multimer |
| 7 | 211960 | SEQ ID NO: 11 × SEQ ID NO: 12 | Dimer |
| 8 | 211944 | SEQ ID NO: 13 × SEQ ID NO: 14 | Dimer |
| 9 | 211828 | SEQ ID NO: 15 × SEQ ID NO: 16 | Dimer |
| 19 | 215192 | SEQ ID NO: 10 × SEQ ID NO: 10 | Dimer |
| 17 | 215194 | SEQ ID NO: 10 × SEQ ID NO: 9 | Trimer |
| 18 | 215193 | SEQ ID NO: 9 × SEQ ID NO: 10 | Trimer |
| 20 | 215191 | SEQ ID NO: 9 × SEQ ID NO: 9 | Tetramer |

Coupling sites. In some embodiments, the PD-L1 binding protein as described herein further comprises one or more coupling site(s) for the coupling of chemical moieties. A coupling site is capable of reacting with other chemical groups to couple the PD-L1 binding protein to chemical moieties. The defined number and defined position of coupling sites enables site-specific coupling of chemical moieties to the PD-L1 binding proteins as described herein. Thus, a large number of chemical moieties can be bound to a PD-L1 binding protein if required. The number of coupling sites can be adjusted to the optimal number for a certain application by a person skilled in the art to adjust the amount of the chemical moieties accordingly. In selected embodiments, the coupling site may be selected from the group of one or more amino acids which can be labeled with specific chemistry such as one or more cysteine residues, one or more lysine residues, one or more tyrosine, one or more tryptophan, or one or more histidine residues. The PD-L1 binding protein may comprise 1 to 20 coupling site(s), preferably 1 to 6 coupling site(s), preferably 2 coupling sites, or preferably one coupling site.

Coupling domains. One embodiment provides a PD-L1 binding protein that comprises at least one coupling domain of 1 to 80 amino acids comprising one or more coupling sites. In some embodiments, the coupling domain of 1 to 80 amino acids may comprise alanine, proline, or serine, and as coupling site cysteine. n other embodiments, the coupling domain of 5 to 80 amino acids may consist of alanine, proline, serine, and as coupling site cysteine. In one embodiment, the coupling domain is consisting of 20-60% alanine, 20-40% proline, 10-60% serine, and one or more cysteine as coupling site(s) at the C- or N-terminal end of the PD-L1 binding protein as described herein. In some embodiments the amino acids alanine, proline, and serine are randomly distributed throughout a coupling domain amino acid sequence so that not more than a maximum of 2, 3, 4, or 5 identical amino acid residues are adjacent, preferably a maximum of 3 amino acids. The composition of the 1 to 20 coupling domains can be different or identical.

In some embodiments, the chemical moieties are selected from any of chelators, drugs, toxins, dyes, and small molecules. In some embodiments, at least one of the chemical moieties is a chelator designed as a complexing agent for coupling one or more further moieties to the targeted compound to the PD-L1 binding protein as disclosed herein. One embodiment relates to a PD-L1 binding protein wherein the chelator is a complexing agent for coupling one or more radioisotopes or other detectable labels.

Diagnostic moiety. In various embodiments, the PD-L1 binding protein further comprises a diagnostic moiety. In other embodiments, the PD-L1 binding protein further comprises more than one diagnostic moiety. In some embodiments, such diagnostic moiety may be selected from radionuclides, fluorescent proteins, photosensitizers, dyes, enzymes, magnetic beads, metallic beads, colloidal particles, electron-dense reagent, biotin, digoxigenin, hapten, or any combination of the above. In some embodiments, a PD-L1 binding protein that comprises at least one diagnostic moiety can be employed, for example, as imaging agents, for example to evaluate presence of tumor cells or metastases, tumor distribution, and/or recurrence of tumor. Methods for detection or monitoring of cancer cells involve imaging methods. Such methods involve imaging PD-L1 related cancer cells by, for example, radioimaging or photoluminescense or fluorescence.

Therapeutic moiety. In various embodiments, the PD-L1 binding protein further comprises a therapeutically active moiety. In other embodiments, the PD-L1 binding protein further comprises more than one therapeutically active moiety. In some embodiments, such therapeutically active moiety may be selected from a monoclonal antibody or a fragment thereof, an extracellular domain of a receptor or fragments thereof, a radionuclide, a cytotoxic compound, a cytokine, a chemokine, an enzyme, CAR-T, or exosomes, or derivatives thereof, or any combination of the above. In some embodiments, the PD-L1 binding protein that comprises a therapeutically active component may be used in targeted delivery of any of the above listed components to the PD-L1 expressing tumor cell and accumulate therein, thereby resulting in low levels of toxicity to normal cells.

Radionuclides. Suitable radionuclides for applications in imaging (for example, in vitro) or for radiotherapy include for example but are not limited to the group of gamma-emitting isotopes, the group of positron emitters, the group of beta-emitters, and the group of alpha-emitters. In some embodiments, suitable conjugation partners include chelators such as 1,4,7,10-tetraazacyclododecane-1,4,7,10-tetraacetic acid (DOTA) or diethylene triamine pentaacetic acid (DTPA) or their activated derivatives, nanoparticles and liposomes. In various embodiments, DOTA may be suitable as complexing agent for radioisotopes and other agents for imaging.

Moiety modulating pharmacokinetics. In some embodiments, the PD-L1 binding protein further comprises at least one moiety modulating pharmacokinetics optionally selected from a polyethylene glycol, a human serum albumin, an albumin-binding peptide, an immunoglobulin binding peptide or an immunoglobulin or immunoglobulin fragments, a sialic acid, or a transferrin, a polysaccharide (for example, hydroxylethyl starch), or an unstructured amino acid sequence which increases the hydrodynamic radius such as a multimer comprising amino acids alanine, glycine, serine, proline. In various embodiments, said moiety increases the half-life of the PD-L1 binding protein at least 1.5 fold. Several techniques for producing PD-L1 binding protein with extended half-life are known in the art, for example, direct fusions of the moiety modulating pharmacokinetics with the PD-L1 binding protein as described above or chemical coupling methods. The moiety modulating pharmacokinetics can be attached for example at one or several sites of the PD-L1 binding protein through a peptide linker sequence or through a coupling site as described above.

Conjugation of proteinaceous or non-proteinaceous moieties to the PD-L1 binding protein may be performed applying chemical methods well-known in the art. In some embodiments, coupling chemistry specific for derivatization of cysteine or lysine residues may be applicable. Chemical coupling can be performed by chemistry well known to someone skilled in the art, including but not limited to, substitution, addition or cycloaddition or oxidation chemistry (e.g. disulfide formation).

Molecules for purification/detection. In some embodiments, additional amino acids can extend either at the N-terminal end of the PD-L1 binding protein or the C-terminal end or both. Additional sequences may include for example sequences introduced e.g. for purification or detection. In one embodiment, additional amino acid sequences include one or more peptide sequences that confer an affinity to certain chromatography column materials. Typical examples for such sequences include, without being limiting, Strep-tags, oligohistidine-tags, glutathione S-transferase, maltose-binding protein, inteins, intein fragments, or the albumin-binding domain of protein G.

Use in medicine. Various embodiments relate to the PD-L1 binding protein as disclosed herein for use in medicine. In one embodiment, the PD-L1 binding protein is used in medicine to diagnose or treat cancer associated with PD-L1 expression. Accordingly, disclosed herein is a method of diagnosis or treatment of PD-L1 related cancer or infectious diseases. The PD-L1 binding proteins as disclosed herein allow selective diagnosis and treatment of PD-L1 related cancer cells or cancer tissues, for example, from melanoma, NSCLC, head and neck cancer, bladder cancer, breast cancer, and others. PD-L1 is known to be upregulated in tumor cells, possibly resulting in uncontrolled growth of tumor cells and in the formation of metastases. The PD-L1 binding proteins as disclosed herein further allow selective diagnosis and treatment of PD-L1 related infectious diseases such as chronic viral infection (HIV, HBV, HCV). In one embodiment, the PD-L1 binding protein is used to diagnose PD-L1 related cancer or infectious diseases by applying in vitro methods.

One embodiment is a method of diagnosing (including monitoring) a subject having PD-L1 related cancer, the method of diagnosis (monitoring) comprising administering to the subject the PD-L1 binding protein as described, optionally conjugated to radioactive molecules. In various embodiments, the PD-L1 binding protein as disclosed herein may be used for diagnosis of PD-L1 related cancer, optionally wherein the PD-L1 binding protein is conjugated to a radioactive molecule. In some embodiments, the PD-L1 binding protein is used in (in vitro) imaging methods with labels such as radioactive or fluorescent and can be employed to visualize PD-L1 on specific tissues or cells, for example, to evaluate presence of PD-L1 related tumor cells, PD-L1 related tumor distribution, recurrence of PD-L1 related tumor, and/or to evaluate the response of a patient to a therapeutic treatment.

One embodiment is a method of treating a subject having PD-L1 related cancer, the method of treatment comprising administering to the subject the PD-L1 specific binding protein as described, optionally conjugated to a radioactive molecule and/or a cytotoxic agent, or as immunooncological agent. In various embodiments, the PD-L1 binding protein as disclosed herein may be used for treatment of PD-L1 related cancer, optionally wherein the PD-L1 binding protein is conjugated to a cytotoxic agent and/or to a radioactive molecule or expressed on the surface of target specific CarT cells. Some embodiments relate to the use of the PD-L1 binding protein labelled with a suitable radioisotope or cytotoxic compound or treatment of PD-L1 related tumor cells, in particular to control or kill PD-L1 related tumor cells, for example malignant cells. In one embodiment, curative doses of radiation are selectively delivered of to PD-L1 related tumor cells but not to normal cells.

As further described herein, in various embodiments, a PD-L1 related cancer is characterized by cancer cells expressing, or overexpressing, PD-L1. In various embodiments, the PD-L1 related cancer is a solid tumor expressing, or overexpressing, PD-L1. A use as immunooncology agent refers to a use of the PD-L1 Affilin® as disclosed herein as therapeutical composition for the treatment of diseases, i.e. to promote an immune response against cancer. Certain cancer cells act to inhibit an immune response against that cell, such as by inhibiting a T cell response against the cell. The PD-L1 specific Affilin® as disclosed herein as part of a medical composition for use as therapeutic agent may counteract the inhibition of the immune response.

Compositions. Various embodiments relate to a composition comprising the PD-L1 binding protein as disclosed herein. A composition comprising the PD-L1 binding protein as defined above for use in medicine, preferably for use in the diagnosis or treatment of various PD-L1 related cancer tumors. Compositions comprising the PD-L1 binding protein as described above may be used for clinical applications for both diagnostic and therapeutic purposes. In particular, compositions comprising the PD-L1 binding protein as described above may be used for clinical applications for imaging, monitoring, and eliminating or inactivating pathological cells that express PD-L1.

Various embodiments relate to a diagnostic composition for the diagnosis of PD-L1 related cancer comprising the PD-L1 binding protein as defined herein and a diagnostically acceptable carrier and/or diluent. These include for example but are not limited to stabilizing agents, surface-active agents, salts, buffers, coloring agents etc. The compositions can be in the form of a liquid preparation, a lyophilisate, granules, in the form of an emulsion or a liposomal preparation.

The diagnostic composition comprising the PD-L1 binding protein as described herein can be used for diagnosis of PD-L1 related cancer, as described above.

Various embodiments relate to a pharmaceutical (e.g. therapeutical) composition for the treatment of diseases comprising the PD-L1 binding protein as disclosed herein, and a pharmaceutically (e.g. therapeutically) acceptable carrier and/or diluent. The pharmaceutical (e.g. therapeutical) composition optionally may contain further auxiliary agents and excipients known per se. These include for example but are not limited to stabilizing agents, surface-active agents, salts, buffers, coloring agents etc.

The pharmaceutical composition comprising the PD-L1 binding protein as defined herein can be used for treatment of diseases, as described above.

The compositions contain an effective dose of the PD-L1 binding protein as defined herein. The amount of protein to be administered depends on the organism, the type of disease, the age and weight of the patient and further factors known per se. Depending on the galenic preparation these compositions can be administered parenterally by injection or infusion, systemically, intraperitoneally, intramuscularly, subcutaneously, transdermally, or by other conventionally employed methods of application.

The composition can be in the form of a liquid preparation, a lyophilisate, a cream, a lotion for topical application, an aerosol, in the form of powders, granules, in the form of an emulsion or a liposomal preparation. The type of preparation depends on the type of disease, the route of administration, the severity of the disease, the patient and other factors known to those skilled in the art of medicine.

The various components of the composition may be packaged as a kit with instructions for use.

Preparation of PD-L1 binding proteins. PD-L1 binding proteins as described herein may be prepared by any of the many conventional and well known techniques such as plain organic synthetic strategies, solid phase-assisted synthesis techniques, fragment ligation techniques or by commercially available automated synthesizers. On the other hand, they may also be prepared by conventional recombinant techniques alone or in combination with conventional synthetic techniques. Furthermore, they may also be prepared by cell-free in vitro transcription/translation.

Various embodiments relate to a polynucleotide encoding a PD-L1 binding protein as disclosed herein. One embodiment further provides an expression vector comprising said polynucleotide, and a host cell comprising said isolated polynucleotide or the expression vector.

Various embodiments relate to a method for the production of a PD-L1 binding protein as disclosed herein comprising culturing of a host cell under suitable conditions which allow expression of said PD-L1 binding protein and optionally isolating said PD-L1 binding protein. For example, one or more polynucleotides which encode for the PD-L1 binding protein may be expressed in a suitable host and the produced PD-L1 binding protein can be isolated. A host cell comprises said nucleic acid molecule or vector. Suitable host cells include prokaryotes or eukaryotes. A vector means any molecule or entity (e.g., nucleic acid, plasmid, bacteriophage or virus) that can be used to transfer protein coding information into a host cell. Various cell culture systems, for example but not limited to mammalian, yeast, plant, or insect, can also be employed to express recombinant proteins. Suitable conditions for culturing prokaryotic or eukaryotic host cells are well known to the person skilled in the art. Cultivation of cells and protein expression for the purpose of protein production can be performed at any scale, starting from small volume shaker flasks to large fermenters, applying technologies well-known to any skilled in the art.

One embodiment is directed to a method for the preparation of a binding protein as detailed above, said method comprising the following steps: (a) preparing a nucleic acid encoding a PD-L1 binding protein as defined herein; (b) introducing said nucleic acid into an expression vector; (c) introducing said expression vector into a host cell; (d) cultivating the host cell; (e) subjecting the host cell to culturing conditions under which a PD-L1 binding protein is expressed, thereby producing a PD-L1 binding protein as defined herein; (f) optionally isolating the PD-L1 binding protein produced in step (e); and (g) optionally conjugating the PD-L1 binding protein with further functional moieties as defined herein.

In general, isolation of purified PD-L1 binding protein from the cultivation mixture can be performed applying conventional methods and technologies well known in the art, such as centrifugation, precipitation, flocculation, different embodiments of chromatography, filtration, dialysis, concentration and combinations thereof, and others. Chromatographic methods are well-known in the art and comprise without limitation ion exchange chromatography, gel filtration chromatography (size exclusion chromatography), hydrophobic interaction chromatography or affinity chromatography.

For simplified purification, the PD-L1 binding protein can be fused to other peptide sequences having an increased affinity to separation materials. Preferably, such fusions are selected that do not have a detrimental effect on the functionality of the PD-L1 binding protein or can be separated after the purification due to the introduction of specific protease cleavage sites. Such methods are also known to those skilled in the art.

### EXAMPLES

The following Examples are provided for further illustration of the invention. The invention is particularly exemplified by modifications of ubiquitin resulting in binding to PD-L1. The invention, however, is not limited thereto, and the following Examples merely show the practicability of the invention on the basis of the above description.

### Example 1. Identification of PD-L1 Binding Proteins

Library construction and cloning of libraries. Different proprietary libraries comprising randomized amino acid positions in ubiquitin and/or inserts were in house synthesized by randomized oligonucleotides generated by synthetic trinucleotide phosphoramidites (ELLA Biotech) to achieve a well-balanced amino acid distribution with simultaneously exclusion of cysteine and other amino acid residues at randomized positions.

The corresponding cDNA libraries were amplified by PCR and ligated with a modified pCD87SA phagemid (herein referred to as pCD12) using standard methods known to a skilled person. The pCD12 phagemid comprises a modified torA leader sequence (deletion of amino acid sequence QPAMA) to achieve protein processing without additional amino acids at the N terminus. Aliquots of the ligation mixture were used for electroporation of E. coli ER2738 (Lucigen). Unless otherwise indicated, established recombinant genetic methods were used. Target. The selection was performed with human PD-L1 protein as target. On the one hand IgG₁-Fc-fused proteins were applied (purchased from R&D; Catalog-Nr.: 156-B7), on the other hand biotinylated AviHis-fused proteins were applied (purchased from AcroBiosystems; Catalog-Nr.: PD-1-H82E5).

Primary selection by TAT Phage Display. The naive libraries were enriched against PD-L1 using phage display as selection system. After transformation of competent bacterial ER2738 cells (Lucigene) with phagemid pCD12 carrying the library, phage amplification and purification was carried out using standard methods known to a skilled person. For selection the target protein was immobilized to magnetic beads. Target proteins fused to IgG₁-Fc were immobilized to Protein A or Protein G Dynabeads®. Site-directed biotinylated target proteins fused to AviHis-tag were immobilized on Streptavidin or Neutravidin SpeedBeads™. The PD-L1 concentration during phage incubation was lowered from 200 nM (first round) to 100 nM (second round). and 10 nM (third round). The selections against IgG1-Fc-fused targtes were performed with only two rounds, including a preselection with Fc-fragments of IgG₁ in round 2. The selection against biotinylated AviHis-fused targets included a preincubation of the phage particles with mouse serum, starting in round 2 for 1 h and 23 h in round 3. A preselection was only performed with empty blocked SpeedBeads™. PD-L1 phage complexes were magnetically separated from supernatant and washed several times. PD-L1 bound phages were eluted by trypsin. To identify target specific phage pools, eluted and reamplified phages of each selection round were analysed by phage pool ELISA. Wells of medium binding microtiter plates (Greiner Bio-One) were coated with PD-L1 (2.5 μg/ml). Biotinylated AviHis-tagged PD-L1 was coated via Streptavidin. Bound phages were detected using α-M13 HRP-conjugated antibody (GE Healthcare).

Cloning of target binding phage pools into an expression vector. Selection pools showing specific binding to PD-L1 in phage pool ELISA were amplified by PCR according to methods known in the art, cut with appropriate restriction nucleases and ligated into a derivative of the expression vector pET-28a (Merck, Germany) comprising a Strep-Tag II (IBA GmbH).

Single colony hit analysis. After transformation of BL21 (DE3) cells (Merck, Germany) kanamycin-resistant single colonies were grown. Expression of the PD-L1-binding proteins was achieved by cultivation in 384 well plates (Greiner Bio-One) using auto induction medium (Studier, 2005, Protein Expr. Purif. 41(1):207-234). Cells were harvested and subsequently lysed mechanically by freeze/thaw cycles, respectively. After centrifugation the resulting supernatants were screened by ELISA with immobilized ProtA/PD-L1-Fc on high binding 384 ELISA microtiter plates (Greiner Bio-One). Detection of protein bound to PD-L1-Fc was achieved by Strep-Tactin® HRP Conjugate (IBA GmbH) in combination with TMB-Plus substrate (Biotrend, Germany). The reaction was stopped by addition of 0.2 M H₂SO₄ solution and measured in a plate reader at 450 nm versus 620 nm. Specific hits were selected for μ-scale purification, SPR analysis, sequencing and further for expression in lab scale.

### Example 2. Expression and Purification of PD-L1 Binding Proteins

The genes for PD-L1 binding proteins were cloned into an expression vector using standard methods known to a skilled person, purified and analyzed as described below. All PD-L1 specific proteins were expressed and highly purified by affinity chromatography and gel filtration. After affinity chromatography purification a size exclusion chromatography (SE HPLC or SEC) was performed using an Äkta system and a Superdex™ 200 HiLoad 16/600 column (GE Healthcare). The column had a volume of 120 ml and was equilibrated with 2 CV. The samples were applied with a flow rate of 1 ml/min purificationusing PBS as running buffer. Fraction collection started as the signal intensity reached 10 mAU. Following SDS-PAGE analysis positive fractions were pooled and their protein concentrations were measured.

Further analysis included SDS-PAGE, SE-HPLC and RP-HPLC. Protein concentrations were determined by absorbance measurement at 280 nm using the specific molar absorbent coefficient. RP chromatography (RP HPLC) was performed using a Dionex HPLC system and a Vydac 214MS54 C4 (4.6×250 mm, 5 μm, 300 Å) column (GE Healthcare).

TABLE 2
Analytical results

| | | | | | | rp-HPLC |
|---|---|---|---|---|---|---|
| SEQ ID | | Soluble | Yield (per | | Yield (per | (main |
| NO: | CID | expression | volume) | | weight) | peak) |
| 1 | 215306 | 0.8 | 16.76 | mg/L | 0.68 mg/g | 0.99 |
| 2 | 215220 | 0.7 | 19.10 | mg/L | 1.68 mg/g | 0.7086 |
| 3 | 211904 | 0.5 | 28.12 | mg/L | 1.21 mg/g | 0.8207 |
| 4 | 215208 | 0.4 | 3.08 | mg/L | 0.15 mg/g | 0.9409 |
| 5 | 211620 | 0.5 | 37.10 | mg/L | 1.64 mg/g | 0.7809 |
| 6 | 215258 | 0.8 | 16.75 | mg/L | 1.13 mg/g | 0.7734 |
| 7 | 211960 | 0.9 | 43.03 | mg/L | 1.91 mg/g | 0.9017 |
| 8 | 211944 | 0.95 | 23.50 | mg/L | 1.00 mg/g | 0.9535 |
| 9 | 211828 | 0.9 | 5.55 | mg/L | 0.24 mg/g | 0.919 |
| 10 | 211883 | 0.6 | 1.98 | mg/L | 0.11 mg/g | 0.8975 |

### Example 3: Mammalian Expression of hPD-L1-Fc and mPD-L1-Fc

Expi293-F-cells were cultured with 0.5-1 Mio cells/ml in Expi293-F Expression medium (Fisher scientific, 13489756) in shake flasks at 135 rpm, 37° C., 8% CO₂ and 95% humidity. 1 day before transfection, cells were seeded with a density of 2.0 Mio cells/ml. On the day of transfection, cells were seeded with a density of 2.5 Mio cells/ml. 1 μg plasmid-DNA of hPD-L1-Fc (SEQ ID NO: 26) or mPD-L1-Fc (SEQ ID NO: 27) per ml of culture volume were diluted in Opti-MEM® | Reduced Serum Medium (Life Technologies, 31985-062). ExpiFectamine™ was diluted in Opti-MEM® | Reduced Serum Medium, according to manufacturer information and incubated for 5 min at rt. Subsequently, DNA-solution was added to the ExpiFectamine-mixture, incubated for 20 min at rt and mixed with the cells. After 16 h Enhancer was added to the cells. Supernatant was collected after 96-120 h, centrifuged and filtered through a 0.45 μm membrane.

### Example 4. PD-L1 Binding Proteins are Stable at High Temperatures (Above 62° C.)

Thermal stability of the PD-L1 specific Affilin® proteins was determined by Differential Scanning Fluorimetry (DSF). Each probe was transferred at concentrations of 0.1 μg/μL to a MicroAmp Optical 384-well plate, and SYPRO Orange dye was added at suitable dilution. A temperature ramp from 25 to 95° C. was programmed with a heating rate of 1° C./min (V2A-7 Applied Biosystems). Fluorescence was constantly measured at an excitation wavelength of 520 nm and the emission wavelength at 623 nm (V2A-7, Applied Biosystems). The midpoints of transition for the thermal unfolding (Tₘ, melting points) were determined and are shown in Table 3. All PD-L1 specific Affilin® proteins are stable at temperatures of at least 60° C.

### Example 5. Analysis of PD-L1 Binding Proteins (Surface Plasmon Resonance, SPR)

A CM5 sensor chip (GE Healthcare) was equilibrated with PBS (0.05% surfactant 20) as SPR running buffer. Surface-exposed carboxylic groups were activated by passing a mixture of EDC and NHS to yield reactive ester groups. 700-1500 RU PD-L1 (on-ligand) were immobilized on a flow cell. A flow cell without ligand was used as reference. Injection of ethanolamine after ligand immobilization was used to block unreacted NHS groups. Upon ligand binding, protein analyte was accumulated on the surface increasing the refractive index. This change in the refractive index was measured in real time and plotted as response or resonance units (RU) versus time. The analytes were applied to the chip in serial dilutions with a flow rate of 30 μl/min. The association was performed for 120 seconds and the dissociation for 240 seconds. After each run, the chip surface was regenerated with 30 μl regeneration buffer (0.1 M glycin pH 2.0) and equilibrated with SPR running buffer. The control samples were applied to the matrix with a flow rate of 30 μl/min, while they associate for 120 seconds and dissociate for 240 seconds. Regeneration and re-equilibration were performed as previously mentioned. Binding studies were carried out by the use of the Biacore 3000 (GE Healthcare); data evaluation was operated via the BIAevaluation 3.0 software, provided by the manufacturer, by the use of the Langmuir 1:1 model (RI=0). Table 3 shows the binding affinity of PD-L1 binding proteins to PD-L1-Fc. The affinity vs. hPD-L1-Fc is maximal 220 nM. Affilin®211883 and Affilin®211828 bind cross-specific to mouse and human PD-L1.

TABLE 3
Binding affinity (K_{D}) of PD-L1 binding
proteins and temperature stability.

| | | Biacore | | DSF |
|---|---|---|---|---|
| SEQ ID | | KD vs hPD- | | Thermal |
| NO: | Affilin @ | L1-Fc | | transition |
| 1 | 215306 | 11 | nM | 70° C. |
| 2 | 215220 | 96 | pM | n.d. |
| 3 | 211904 | 4 | nM | n.d. |
| 4 | 215208 | 2 | nM | 70° C. |
| 5 | 211620 | 15 | nM | n.d. |
| 6 | 215258 | 100 | nM | n.d. |
| 7 | 211960 | 220 | nM | 64° C. |
| 8 | 211944 | 50 | nM | 61° C. |
| 9 | 211828 | 84 | nM | 71° C. |
| 10 | 211883 | 91 | nM | 62° C. |
| 17 | 215194 | 10 | nM | 64° C. |
| 18 | 215193 | 10 | nM | 65° C. |
| 19 | 215192 | 8 | nM | 69° C. |
| 20 | 215191 | 13 | nM | 69° C. |

### Example 6. Analysis of Competitive Binding to PD-1/PD-L1 Interface

Competitive binding of Affilin®211828 or Affilin®211883 to hPD-1/hPD-L1-Fc was investigated as followed: hPD-1 was immobilized on a CM5 Biacore chip and hPD-L1-Fc was injected with or w/o equimolar addition Affilin®211828 or Affilin®211883.

Competitive binding of Affilin®211904 to hPD-1/hPD-L1-Fc was investigated as followed: PD-L1-Fc fusion protein (15 μl; 30 nM) was immobilized on a CM5 Biacore chip that was coupled with recombinant Protein A using NHS/EDC chemistry resulting in 450 response units (RU). In a first Experiment, 211904 was injected at one defined concentration (50 μL; 5 μM) at a flow of 30 μl/min PBST 0.05% Tween 20. In the next step, 1 μM hPD-1 was applied to the same flow cell. Alternatively, in the second experiment, the same flow channel was first pre-loaded with 50 μL PBST 0.05% Tween20 followed by loading of 1 μM hPD-1. Results: Affilin®211828, Affilin®211883, and Affilin®211904 showed competitive binding to human PD-1/PD-L1 interface; the addition of Affilin® proteins leads to significant reduced binding of hPD-L1-Fc to hPD-1. Further, Affilin®211828 and Affilin®211883 were tested for binding of mouse PD-L1-Fc to mouse PD-1. The results were comparable to the competitive binding to human PD-1/PD-L1 interface.

### Example 7. Competition of Binding Proteins for Epitopes

Competitive binding of two isolated Affilin®-proteins to hPD-L1-Fc (203395) was investigated as followed: the first Affilin®-protein was immobilized on a CM5 Biacore chip (˜100 RU) and hPD-L1-Fc was injected with or without equimolar or five-fold excess of the second Affilin® protein.

For Affilin®211904, the competitive binding with other Affilin® proteins to hPD-L1-FC was investigated as followed: PD-L1-Fc fusion protein (15 μL; 30 nM) was immobilized on a CM5 Biacore chip that was coupled with recombinant Protein A using NHS/EDC chemistry resulting in ˜450 response units (RU). In a first experiment, Affilin®211904 was injected at one defined concentration (5 μM) at a flow of 5 μl/min PBST 0.05% Tween 20 until chip surface was saturated. In the next step, 60 μL of a second Affilin® (5 μM) was applied to the same flow channel In the second experiment, the same flow channel was first pre-loaded with 60 μL PBST 0.05% Tween 20 followed by loading of 60 μL second Affilin® (5 μM).

Results are shown in Table 4. In Table 4, “comp” means that the binding of the first Affilin® was influenced by the presence of the second Affilin®, and vice versa. All PD-L1 specific Affilin® proteins bind to the same or overlapping epitopes, i.e. to the same or overlapping surface exposed amino acids. “n.d.” refers to not determined.

TABLE 4
Competition of Affilin ® proteins for epitopes

| | | SEQ ID NO: | | | | | | | | |
|---|---|---|---|---|---|---|---|---|---|---|
| | | | | | | | | 20 | | 19 |
| | | | | | | | | 215191 | | 215192 |
| | | | | | | | | (Dimer | | (Dimer |
| | | 1 | 2 | 4 | 5 | 8 | 9 | of | 10 | of |
| | Affilin ® | 215306 | 215220 | 215208 | 211620 | 211944 | 211828 | 211828) | 211883 | 211883) |
| 2 | 215220 | comp | comp | n.d. | comp | comp | comp | n.d. | comp | n.d. |
| 3 | 211904 | comp | comp | comp | comp | comp | n.d. | comp | n.d | comp |
| 5 | 211620 | comp | comp | n.d. | comp | comp | comp | n.d. | comp | n.d. |
| 8 | 211944 | comp | comp | n.d. | comp | comp | comp | n.d. | comp | n.d. |
| 9 | 211828 | comp | comp | n.d. | comp | comp | comp | n.d. | comp | n.d. |
| 10 | 211883 | comp | comp | n.d. | comp | comp | comp | n.d. | comp | n.d. |

### Example 8. Functional Characterization: Specific Binding to Cell Surface Expressed hPD-L1 and mPD-L1 (Flow Cytometry)

Flow cytometry was used to analyze the specific interaction of PD-L1 binding proteins with surface-exposed hPD-L1 or mPD-L1. Transfected HEK293-hPD-L1-cells, HEK293-mPD-L1-cells, empty vector control HEK293-pEntry-cells and the native hPD-L1-expressing H460-cell line were trypsinized and resuspended in medium containing FCS and washed in pre-cooled FACS blocking buffer. A cell concentration of 1 Mio cells/ml was prepared for cell staining and filled with 100 μl/well into a 96 well plate (Greiner) in triplicate for each cell line. A dilution series of Affilin proteins or 1 μg/ml Avelumab (Merck) as positive control was added to PD-L1-expressing and control cells. After 45 min the supernatants were removed, and 100 μl/well rabbit anti-Strep-Tag antibody (GenScript; A00626), 1:300 diluted in FACS blocking buffer, were added. Avelumab was detected with anti-human-IgG-Alexa 488 (Invitrogen; A-11013) with a dilution of 1:1000. After removal of the primary antibody, goat anti-rabbit IgG Alexa Fluor 488 antibody (Invitrogen; A11008) was applied in a 1:1000 dilution to the other wells. Flow cytometry measurement was conducted on the Guava easyCyte 5HT device (Merck-Millipore) at excitation wavelength 488 nm and emission wavelength 520 nm.

All Affilin® proteins showed specific binding on hPD-L1-overexpressing HEK293-cells, but no binding on HEK293-pEntry-cells (data not shown). Affilin®211620, Affilin®211904, Affilin®211944, Affilin®215208 and Affilin®215258 show also a significant binding (high affinity binding) to native hPD-L1-expressing cells (Table 5). Affilin®211828 and Affilin®211838 show binding both on hPD-L1 and mPD-L1-expressing cells (Table 6).

TABLE 5
Cell binding analysis of Affilin ® proteins on HEK293-hPD-
L1-overexpressing cells and H460 cell line (Flow cytometry)

| SEQ ID NO: | Affilin ® | HEK293-hPDL1 1 μM | H460 1 μM |
|---|---|---|---|
| 3 | 211904 | 40.97 | 18.03 |
| 4 | 215208 | 321.77 | 10.22 |
| 5 | 211620 | 95.3 | 6.1 |
| 6 | 215258 | 289.17 | 3.98 |
| 7 | 211944 | 27.67 | 3.39 |

TABLE 6
Crossreactive cell binding on HEK293-
PD-L1-cells (mouse or human)

| | | HEK293-hPDL1 | HEK293-mPDL1 |
|---|---|---|---|
| SEQ ID NO: | Affilin ® | 1 μM | 1 μM |
| 9 | 211828 | 12.84 | 12.78 |
| 10 | 211883 | 7.43 | 3.96 |

### Example 9. Serum Stability of PD-L1 Specific Affilin® Proteins (Cell Binding Assay—Flow Cytometry)

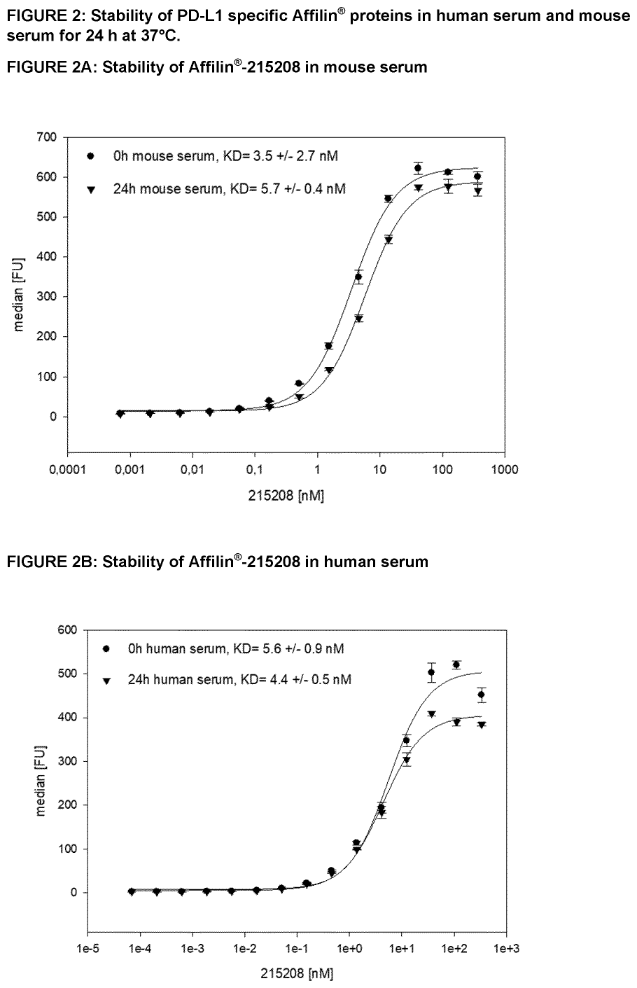

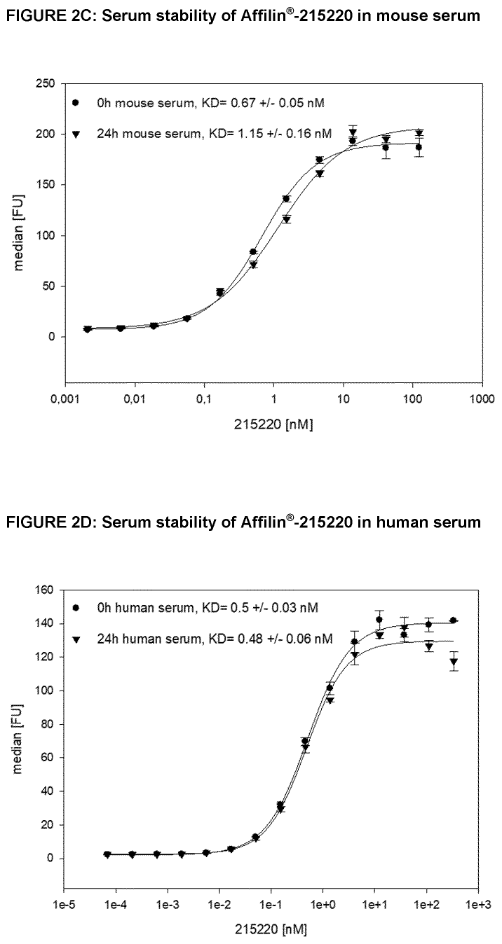

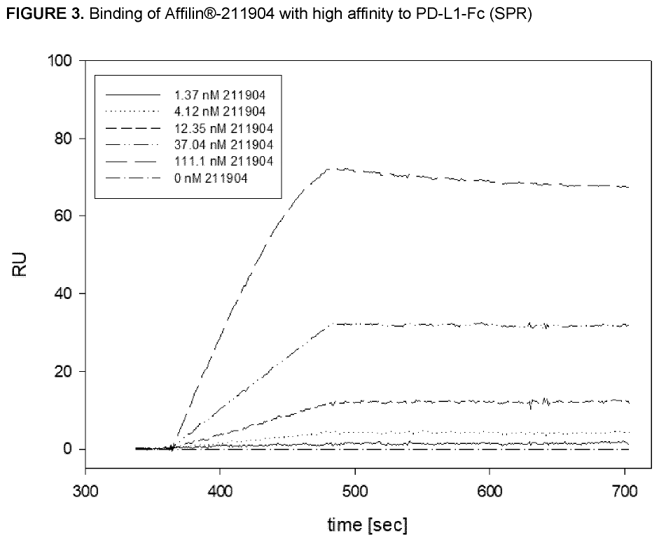

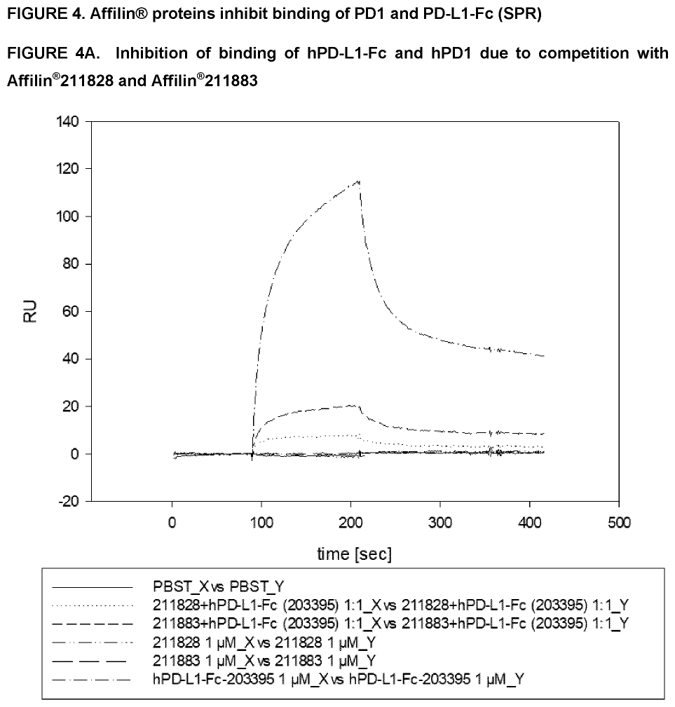

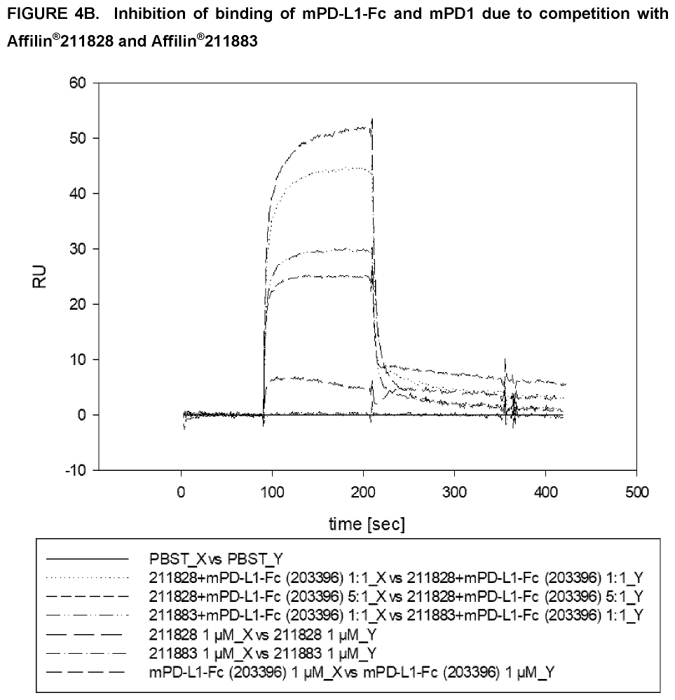

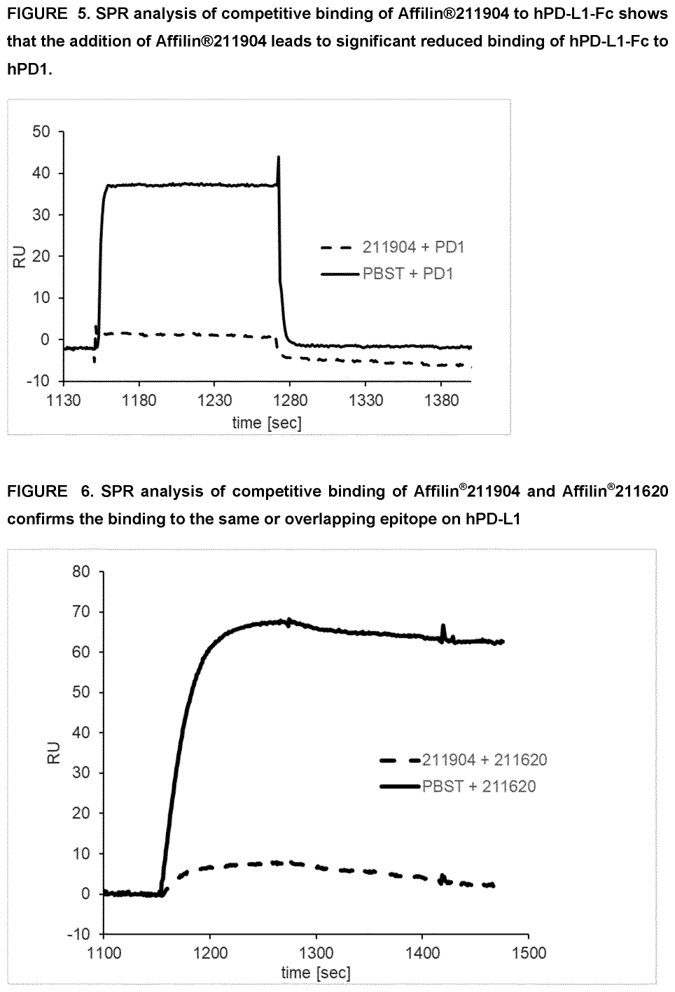

Dilution series from 370 nM to 70 μM of Affilin®215208 (SEQ ID NO: 4) and 123 nM to 70 μM Affilin®215220 (SEQ ID NO: 2) were incubated in mouse serum and a dilution series of 333 nM to 0.7 nM of Affilin®215208 and Affilin®215220 were incubated in human serum for 24 h at 37° C. PD-L1 overexpressing HEK293-cells were trypsinyzed, washed with FACS blocking buffer, seeded in 96-well round bottom plates with a density of 0.1 Mio cells/100 μl and dilution series of Affilin proteins were incubated after 24 h incubation or 0 h (control) in the presence of serum with the cells. After 45 min the supernatants were removed, and cells were washed. The binding of Affilin® proteins was proven with 100 μl/well rabbit anti-Strep-Tag antibody 1:300 diluted in FACS blocking buffer in a first step and goat anti-rabbit IgG Alexa Fluor 488 antibody in a 1:1000 dilution in a second step. The read out was described below. Both Affilin® molecules are stable in human serum and mouse serum for at least 24 h (FIG. 2A-2D). Affilin®215208 shows a K_{D} of 3.5-5.7 nM. The binding of Affilin®215220 to PD-L1-expressing cells remains very strong even after 24 h incubation in human serum (K_{D} is 400 μM).

1. A protein comprising an amino acid sequence having at least 90% identity to any one of SEQ ID NOs: 1 to 20, wherein the protein has a specific binding affinity for human Programmed Death Ligand 1 (PD-L1) of at least 250 nM, and further wherein the protein inhibits interaction of PD-L1 with a programmed cell death 1 (PD1) polypeptide.

2. The protein according to claim 1, wherein the protein has a binding affinity for human PD-L1 of at least 100 nM.

3. The protein according to claim 1, wherein protein is stable in serum-after for at least 24 hours at 37° C.

4. The protein according to claim 1, wherein the protein is a multimer comprising a plurality of the proteins according to claim 1.

5. The protein according to claim 4, wherein the protein is a dimer, a trimer, or a tetramer of the protein according to claim 1.

6. The protein according to claim 1, further comprising one or more coupling sites for coupling of chemical moieties.

7. The protein according to claim 6, wherein the chemical moieties are selected from the group consisting of chelators, drugs, toxins, dyes, and small molecules.

8. The protein according to claim 1, further comprising at least one diagnostically active moiety.

9. The protein according to claim 8, wherein the at least one diagnostically active moiety is selected from the group consisting of a radionuclide, a fluorescent protein, a photosensitizer, a dye, an enzyme, a magnetic bead, a metallic bead, a colloidal particle, an electron to dense reagent, biotin, digoxigenin, a hapten, a CAR-T, and an exosome, and combinations thereof.

10. The protein according to claim 1, further comprising at least one therapeutically active moiety.

11. The protein according to claim 10, wherein the at least one therapeutically active moiety is selected from the group consisting of a monoclonal antibody or a fragment thereof, a binding protein, a receptor or receptor domain, a receptor ligand, a radionuclide, a cytotoxic compound, a cytokine, a chemokine, an enzyme, a CAR-T, and an exosome, or derivatives thereof, or any combination of the above.

12. The protein according to claim 1, further comprising at least one moiety modulating pharmacokinetics.

13. The protein according to claim 12, wherein the at least one moiety modulating pharmacokinetics is selected from the group consisting of a serum albumin, an albumin binding protein, an immunoglobulin binding protein, an immunoglobulin or immunoglobulin fragment, a polysaccharide, an unstructured amino acid sequence comprising amino acids alanine, glycine, serine, and proline, a polyethylene glycol, a sialic acid, and a transferrin.

14.-16. (canceled)

17. A method for producing a protein according to claim 1, comprising: (a) culturing a host cell under conditions suitable to obtain said protein; and (b) isolating said protein produced from the host cell or from a medium in which the host cell was cultured.

18. A method for detecting PD-L1 in a sample comprising contacting the sample with a protein according to claim 1 and detecting binding of the protein according to claim 1 to PD-L1 present in the sample, whereby PD-L1 in the sample is detected.

19. A method for diagnosing or treating a PD-L1-related tumor or an infectious disease, the method comprising administering to a subject in need thereof the protein accordingly to claim 1 via in an amount and via a route sufficient to diagnose or treat the PD-L1-related tumor or the infectious disease, optionally wherein the infectious disease is a chronic viral infection.

20. A host cell comprising a polynucleotide that encodes the protein of claim 1 or an expression construct that directs expression of the protein of claim 1 in the host cell.


SEQUENCE LISTING (26 sequences)

SEQ ID NO: 1 - protein, 82 aa, Artificial Sequence
  PDL1 binding protein Affilin215306
MQIFVKTLTEQAQEHGKTITLEVEPSDTIENVKAKIQDKEGIPPDQQRLIWAGKQLEDGRTLSDYNIPYWWPLFLFLGLRAA

SEQ ID NO: 2 - protein, 82 aa, Artificial Sequence
  PDL1 binding protein Affilin215220
MQIFVKTLTFKIIPAGKTITLEVEPSDTIENVKAKIQDKEGIPPDQQRLIWAGKQLEDGRTLSDYNIDWPAMLTLSLPLRAA

SEQ ID NO: 3 - protein, 80 aa, Artificial Sequence
  PDL1 binding protein Affilin211904
MQIFVKTLTVLEMGKTITLEVEPSDTIENVKAKIQDKEGIPPDQQRLIWAGKQLEDGRTLSDYNIHSFPMLELLLTLRAA

SEQ ID NO: 4 - protein, 80 aa, Artificial Sequence
  PDL1 binding protein Affilin215208
MQIFVKTLTVYEVGKTITLEVEPSDTIENVKAKIQDKEGIPPDQQRLIWAGKQLEDGRTLSDYNIFALPMLELLLTLRAA

SEQ ID NO: 5 - protein, 76 aa, Artificial Sequence
  PDL1 binding protein Affilin211620
MQIFVWTIITKRITLEVEPSDTIENVKAKIQDKEGIPPDQQHLVWEGKQLEDGRTLSDYNIEKYDYLFLNLHLRAA

SEQ ID NO: 6 - protein, 76 aa, Artificial Sequence
  PDL1 binding protein Affilin215258
MQIFVVTEEDKVITLEVEPSDTIENVKAKIQDKEGIPPDQQMLHWSGKQLEDGRTLSDYNIFDAKILLLHLILRAA

SEQ ID NO: 7 - protein, 152 aa, Artificial Sequence
  PDL1 binding protein Affilin211960
MQIFVQTDTGKTITLEVEPSDTIENVKAKIQDKEGIPPDQQRLIWAGKQLEDGRTLSDYNIGIVAPLHLVLRLRAAMQIFVFTWTGKTITLEVEPSDTIENVKAKIQDKEGIPPDQQRLIWAGKQLEDGRTLSDYNIGNGWMLHLVLRLRAA

SEQ ID NO: 8 - protein, 152 aa, Artificial Sequence
  PDL1 binding protein Affilin211944
MQIFVFTATGKTITLEVEPSDTIENVKAKIQDKEGIPPDQQRLIWAGKQLEDGRTLSDYNIRSGWMLHLVLRLRAAMQIFVATYTGKTITLEVEPSDTIENVKAKIQDKEGIPPDQQRLIWAGKQLEDGRTLSDYNIHEPETLHLVLRLRAA

SEQ ID NO: 9 - protein, 152 aa, Artificial Sequence
  PDL1 binding protein Affilin211828
MQIFVDTLTGKTITLEVEPSDTIENVKAKIQDKEGIPPDQQRLIWAGKQLEDGRTLSDYNIRYPAFLHLVLRLRAAMQIFVRTTTGKTITLEVEPSDTIENVKAKIQDKEGIPPDQQRLIWAGKQLEDGRTLSDYNIWPRRLLHLVLRLRAA

SEQ ID NO: 10 - protein, 76 aa, Artificial Sequence
  PDL1 binding protein Affilin211883
MQIFVKTLTGKTITLAVKPNDTILKVKVWIEIKEGIPPDQQRLIWAGKQLEDGRTLSDYNIQKESTLHLVLRLRAA

SEQ ID NO: 11 - protein, 76 aa, Artificial Sequence
  PDL-1 binding protein (monomer 1 of Affilin(r)211960)
MQIFVQTDTGKTITLEVEPSDTIENVKAKIQDKEGIPPDQQRLIWAGKQLEDGRTLSDYNIGIVAPLHLVLRLRAA

SEQ ID NO: 12 - protein, 76 aa, Artificial Sequence
  PDL-1 binding protein (monomer 2 of Affilin(r)211960)
MQIFVFTWTGKTITLEVEPSDTIENVKAKIQDKEGIPPDQQRLIWAGKQLEDGRTLSDYNIGNGWMLHLVLRLRAA

SEQ ID NO: 13 - protein, 76 aa, Artificial Sequence
  PDL-1 binding protein (monomer 1 of Affilin(r)211944)
MQIFVFTATGKTITLEVEPSDTIENVKAKIQDKEGIPPDQQRLIWAGKQLEDGRTLSDYNIRSGWMLHLVLRLRAA

SEQ ID NO: 14 - protein, 76 aa, Artificial Sequence
  PDL-1 binding protein (monomer 2 of Affilin(r)211944)
MQIFVATYTGKTITLEVEPSDTIENVKAKIQDKEGIPPDQQRLIWAGKQLEDGRTLSDYNIHEPETLHLVLRLRAA

SEQ ID NO: 15 - protein, 76 aa, Artificial Sequence
  PDL-1 binding protein (monomer 1 of Affilin(r)211828)
MQIFVDTLTGKTITLEVEPSDTIENVKAKIQDKEGIPPDQQRLIWAGKQLEDGRTLSDYNIRYPAFLHLVLRLRAA

SEQ ID NO: 16 - protein, 76 aa, Artificial Sequence
  PDL-1 binding protein (monomer 2 of Affilin(r)211828)
MQIFVRTTTGKTITLEVEPSDTIENVKAKIQDKEGIPPDQQRLIWAGKQLEDGRTLSDYNIWPRRLLHLVLRLRAA

SEQ ID NO: 17 - protein, 248 aa, Artificial Sequence
  PDL1 binding protein - fusion protein CID215194 (Affilin211883 fused to Affilin211828)
MQIFVKTLTGKTITLAVKPNDTILKVKVWIEIKEGIPPDQQRLIWAGKQLEDGRTLSDYNIQKESTLHLVLRLRAAGGGGSGGGGSGGGGSGGGGSMQIFVDTLTGKTITLEVEPSDTIENVKAKIQDKEGIPPDQQRLIWAGKQLEDGRTLSDYNIRYPAFLHLVLRLRAAMQIFVRTTTGKTITLEVEPSDTIENVKAKIQDKEGIPPDQQRLIWAGKQLEDGRTLSDYNIWPRRLLHLVLRLRAA

SEQ ID NO: 18 - protein, 248 aa, Artificial Sequence
  PDL1 binding protein - fusion protein CID215193 (Affilin211828 fused to Affilin211883)
MQIFVDTLTGKTITLEVEPSDTIENVKAKIQDKEGIPPDQQRLIWAGKQLEDGRTLSDYNIRYPAFLHLVLRLRAAMQIFVRTTTGKTITLEVEPSDTIENVKAKIQDKEGIPPDQQRLIWAGKQLEDGRTLSDYNIWPRRLLHLVLRLRAAGGGGSGGGGSGGGGSGGGGSMQIFVKTLTGKTITLAVKPNDTILKVKVWIEIKEGIPPDQQRLIWAGKQLEDGRTLSDYNIQKESTLHLVLRLRAA

SEQ ID NO: 19 - protein, 172 aa, Artificial Sequence
  PDL1 binding protein - fusion protein CID215192 (Affilin211883 fused to Affilin211883)
MQIFVKTLTGKTITLAVKPNDTILKVKVWIEIKEGIPPDQQRLIWAGKQLEDGRTLSDYNIQKESTLHLVLRLRAAGGGGSGGGGSGGGGSGGGGSMQIFVKTLTGKTITLAVKPNDTILKVKVWIEIKEGIPPDQQRLIWAGKQLEDGRTLSDYNIQKESTLHLVLRLRAA

SEQ ID NO: 20 - protein, 324 aa, Artificial Sequence
  PDL1 binding protein - fusion protein CID215191 (Affilin211828 fused to Affilin211828)
MQIFVDTLTGKTITLEVEPSDTIENVKAKIQDKEGIPPDQQRLIWAGKQLEDGRTLSDYNIRYPAFLHLVLRLRAAMQIFVRTTTGKTITLEVEPSDTIENVKAKIQDKEGIPPDQQRLIWAGKQLEDGRTLSDYNIWPRRLLHLVLRLRAAGGGGSGGGGSGGGGSGGGGSMQIFVDTLTGKTITLEVEPSDTIENVKAKIQDKEGIPPDQQRLIWAGKQLEDGRTLSDYNIRYPAFLHLVLRLRAAMQIFVRTTTGKTITLEVEPSDTIENVKAKIQDKEGIPPDQQRLIWAGKQLEDGRTLSDYNIWPRRLLHLVLRLRAA

SEQ ID NO: 21 - protein, 76 aa, Artificial Sequence
  ubiquitin
MQIFVKTLTGKTITLEVEPSDTIENVKAKIQDKEGIPPDQQRLIFAGKQLEDGRTLSDYNIQKESTLHLVLRLRAA

SEQ ID NO: 22 - protein, 6 aa, Artificial Sequence
  insert in position 9-10 of SEQ ID NO 1
EQAQEH

SEQ ID NO: 23 - protein, 6 aa, Artificial Sequence
  insert of SEQ ID NO 2 (between pos 9 and 10)
FKIIPA

SEQ ID NO: 24 - protein, 4 aa, Artificial Sequence
  insert of SEQ ID NO 3 (insertion between pos. 9 / 10)
VLEM

SEQ ID NO: 25 - protein, 4 aa, Artificial Sequence
  insert of SEQ ID NO 4 (insertion between pos 9/10)
VYEV

SEQ ID NO: 26 - protein, 220 aa, Artificial Sequence
  ECR of human PD-L1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER